(19) United States
(12) Patent Application Publication
(10) Pub. No.: US 2007/0259367 A1
(43) Pub. Date: Nov. 8, 2007

(54) METHOD TO DIAGNOSE AND INCREASE FERTILITY OF MAMMALIAN SEMEN USING DNASE AS A DIAGNOSTIC MARKER AND THERAPEUTIC AGENT

(76) Inventors: Roy L. Ax, Tucson, AZ (US); Tod C. McCauley, Tucson, AZ (US)

Correspondence Address: Intellectual Property Department;DEWITT ROSS & STEVENS S.C., Excelsior Financial Centre, 8000 Excelsior Drive, Suite 401, Madison, WI, 53717-1914, US

(21) Appl. No.: 11/743,989

(22) Filed: May 3, 2007

Related U.S. Application Data
(60) Provisional application No. 60/797,254, filed on May 3, 2006.

(51) Int. Cl. C12Q 1/68

(52) U.S. Cl. 435/6

(57) ABSTRACT

A method and a corresponding composition of matter for diagnosing or increasing the fertility of semen is described. The method involves measuring DNase activity in a semen sample, wherein greater DNase activity in the semen sample indicates increased fertility of the semen sample. Also described is a corresponding composition for storing and increasing the fertility of semen. The composition includes a diluent and an amount of exogenous DNase and-or fertility-associated antigen (FAA) disposed in the diluent. The amount of the exogenous DNase and/or FAA added to the diluent is effective to increase fertility of the semen stored in the composition.

### CROSS-REFERENCE TO RELATED APPLICATIONS

Priority is hereby claimed to provisional application Ser. No. 60/797,254, filed May 3, 2006, which is incorporated herein by reference.

### BIBLIOGRAPHY

Complete bibliographical citations to the documents cited herein can be found in the Bibliography, immediately preceding the claims.

### FIELD OF THE INVENTION

The present invention is directed to methods and corresponding compositions of matter that utilize DNase as a marker to diagnose and as an agent to enhance the fertility of mammalian semen.

### DESCRIPTION OF THE PRIOR ART

Neutrophils are recruited into the female reproductive tract in response to insemination. This inflammatory response is important for the continued health of the female reproductive tract. The inflammatory response functions to remove excess spermatozoa and microbial contaminants that enter the reproductive tract during the breeding process. See Kaeoket et al., 2003; Tremellen et al., 1998; Troedsson et al., 1998, 2001; and Rozeboom et al., 1998. Notably, however, the presence of neutrophils in the female reproductive tract at the time of insemination has been shown to result in formation of extensive DNA clusters. (Alghamdi et al., 2001; Rozeboom et al., 2001; Alghamdi et al., 2004.) These DNA clusters bind to spermatozoa and reduce their motility (Alghamdi et al., 2001) and their fertility (Alghamdi et al., 2004 and Rozeboom et al., 2000).

Neutrophils exhibit a unique mechanism by which they extrude their nuclear DNA and associated proteins to form neutrophil extracellular traps (“NETs”) that ensnare and kill foreign objects (Brinkmann et al., 2004). Bacteria, yeast, and sperm have all been shown to activate neutrophils, thereby causing a release of nuclear DNA and the formation of NETs. See Brinkmann et al., 2004; Urban et al., 2006; and Alghamdi & Foster, 2005, respectively. Neutrophils in the process of forming NETs exhibit several key features: (1) the formation of NETs is observed in motile neutrophils; (2) NET formation is faster than the apoptosis time-course; (3) NET formation is not accompanied by cytoplasmic markers; and (4) NET-forming neutrophils exclude vital dyes. From these four observations it can be concluded that the NET-forming process is not a result of cell death (Brinkmann et al., 2004).

DNase activity has been identified in semen from several mammalian species. See McCauley et al., 1999; Yasuda et al., 1993; Shastina et al., 2003; and Sato et al., 2003). It has also been shown that DNase activity reduces the formation of sperm-neutrophil clusters (Alghamdi et al., 2005). Artificial insemination breeding trials have been conducted in pigs and horses wherein seminal plasma was deposited simultaneously with the semen in the presence of neutrophils. See Rozeboom, 2000; and Alghamdi, 2004. Fertility was higher as a result of adding seminal plasma in those experiments.

The mechanism underlying the ability of seminal plasma to improve fertility, however, is not known. Alghamdi & Foster (2005) have proposed a model: When sperm are deposited in the female reproductive tract, neutrophils migrate to that site and become activated. Upon activation, those neutrophils release their DNA, setting up a fibrous web to entangle sperm for phagocytosis. Crude seminal plasma protein extract has been shown to reduce sperm-neutrophil binding in vitro in a dose-dependent manner by hydrolyzing the NETs. Again, see Alghamdi & Foster (2005). Thus, seminal plasma contains a component with DNase activity which countervails the negative impact of neutrophils on sperm.

Fertility-associated antigen (FAA) serves as a biomarker for bull fertility. FAA is produced in the seminal vesicles, prostate, and Cowper's glands. FAA binds to sperm as they traverse the male reproductive tract during ejaculation. (McCauley et al., 1999.)

### SUMMARY OF THE INVENTION

The present invention is a method for gauging and improving the fertility of semen from any mammalian species. The method comprises screening a semen sample for DNase activity. The higher the activity of DNase found in a sample, the higher the fertility of that sample. Conversely, the lower the activity of DNase found in the sample, the lower the fertility of that sample. Thus, the method provides a means to rank the fertility of a male of any mammalian species, including humans.

The present invention is also directed to a therapeutic measure to fortify semen with DNase or a DNase I-like protein so that the activity of DNase within the semen can be elevated, thereby improving the fertility of the semen. In particular, increased DNase activity leads to improved fertility at the time of artificial insemination. Thus, semen-containing compositions for use in artificial insemination that are supplemented with exogenous DNase are highly useful to improve the success rate of artificial insemination protocols. The artificial insemination industry is global, fiercely competitive, growing, and encompasses a host of mammals, including humans, bovines, equines, ovines, swine, and endangered mammals such as non-human primates, elephants, rhinos, and the like. Thus, any method or composition that improves the fertility of the semen collected is highly coveted and manifestly useful.

Summarizing, the present invention is directed to the following:

(1) an assay for DNase activity in semen to measure the fertility potential of individual mammalian males. DNase activity correlates positively with fertility;

(2) fortification of semen with DNase or a DNase I-like protein (whether purified or genetically engineered) to improve the fertility of semen as compared to non-treated semen.

(3) therapeutic treatment of sperm with DNase or a DNase I-like protein prior to cryopreservation to improve sperm function, including fertility (but not exclusively limited to fertility).

Thus, a first version of the invention is directed to a method for measuring mammalian semen fertility. The method comprises measuring DNase activity in a semen sample. Increased DNase activity in the semen sample as compared to a control sample indicates increased fertility of the semen sample. The DNase activity can be measured using any means now known in the art or developed in the future for ascertaining DNase activity. See the Examples for exemplary methods. One approach for measuring the DNase activity in the semen sample is to concentrate seminal plasma proteins from the sample to yield seminal plasma protein extract. The seminal plasma protein extract is then spiked with a known amount of DNA to yield a DNA-spiked seminal plasma protein extract. DNA hydrolysis in the DNA-spiked seminal plasma protein extract is then measured (typically by gel electrophoresis). An increased level of DNA hydrolysis indicates an increased level of DNase activity in the semen sample (and thus increased fertility in the semen sample).

If desired, specific types of DNase activity may be measured (as contrasted to overall hydrolysis of DNA in the sample caused by any and all DNases). Thus, the activity of DNase I enzymes can be measured, or DNase I-like proteins can be measured, etc.

Another version of the invention is a method for enhancing the fertility of mammalian semen. Here, the method comprises adding exogenous DNase and/or fertility-associated antigen (FAA) to semen. The treated semen may then be immediately used in artificial insemination, or the treated semen may be cryopreserved.

The amount of added DNase and/or FAA will very based upon the type of semen being treated, whether the semen is to be used immediately or cryopreserved for future use, the concentration of sperm within the semen, prior or subsequent treatments of the semen (e.g., upstream or downstream treatments to sex the sperm contained within the semen), the animal from which the semen is derived, the health, age, and condition of the animal from which the semen sample is taken, etc. Preferably, from about 0.001 μg/mL to about 1.0 μg/mL of exogenous DNase is added to the semen. If FAA is used, from about 10 to about 100 μg/mL of exogenous FAA is added to the semen. Amounts above and below this range are within the scope of the invention, however. The amount of DNase to be added to the semen is ultimately up to the choice of the herd manager or veterinarian.

Preferably, the exogenous DNase added to the semen is a DNase I enzyme, EC 3.1.21.1, or a DNase I-like protein, EC 3.1.21.x.

Another version of the invention is a method of storing semen for future use in artificial insemination. This version of the invention comprises adding a fertility-enhancing-effective amount of exogenous DNase and/or FAA to the semen, and then cryopreserving the semen. As noted earlier, it is preferred that from about 0.001 μg/mL to about 1.0 μg/mL of the exogenous DNase and/or FAA is added to the semen, although concentrations above and below this preferred range are within the scope of the invention. Likewise, it is preferred that the exogenous DNase added to the semen is a DNase I enzyme, EC 3.1.21.1, or a DNase I-like protein, EC 3.1.21.x.

The semen is preferably disposed in a cryopreservation media prior to cryopreservation. Any suitable cryopreservation media may be used, such as citrate-based milk extenders and Tris-based egg-yolk extenders. A host a suitable extenders for mammalian semen are commercially available.

The invention is also directed to a composition of matter for increasing the fertility of semen. The composition comprises a diluent (such as TALP media) and an amount of exogenous DNase and/or exogenous FAA is disposed in the diluent. The amount of the exogenous DNase is effective to increase fertility of the semen. The preferred exogenous DNase is a DNase I enzyme, EC 3.1.21.1 or a DNase I-like protein, EC 3.1.21.x. The preferred concentrations of the added DNase and/or FAA are as noted previously. The composition may comprise semen alone (without sperm) or semen with sperm disposed therein.

The compositions can take the form of storage media or “extenders,” a term of art in the artificial insemination field which refers to any composition used to extend the life of sperm within the semen prior to artificial insemination.

The present invention is also directed to a corresponding method of contraception. In this version of the invention, rather than increasing the activity of DNase in the semen (thereby to increase fertility), the activity of DNase is purposefully inhibited using a DNase-specific inhibitor (preferably a DNase-specific antibody).

Further still, the invention is directed to a method of treating uterine disorders in general and endometritis in particular. The method can be used in all mammals, including livestock and primates (including humans). The method comprises administering a NET-degrading-effective amount of a DNase and/or FAA to the interior of the uterus. Endometritis (for example) is accompanied by the formation of DNA-rich extracellular traps released by neutrophils. DNase activity functions to degrade the NETs, thereby reducing the symptoms of uterine disorder caused by inflammation and the concomitant for formation of NETs. Using DNases and/or FAA degrades NETs within the uterus, thereby cleansing the female reproductive tract, without compromising the bactericidal activity antibacterial neutrophils.

The objects and advantages of the invention will appear more fully from the following detailed description of the preferred embodiment of the invention made in conjunction with the accompanying drawings.

### BRIEF DESCRIPTION OF THE DRAWINGS

FIG. 1 is a gel depicting DNase activity among SP-protein extracts isolated from four (A, B, C, D) individual bulls. One (1) μg of plasmid DNA (pBR322) was incubated with DNAse-I (0.25 μg/ml; positive control) or seminal plasma extracts (1 mg/ml) from each bull. Control represents uncut plasmid DNA. Variation in DNase activity among the four samples is clearly shown based on the differential amount of DNA detected following treatment. The far left lane is a 1 kb DNA molecular weight marker (New England BioLabs, Ipswich, Mass.).

FIG. 2 is a gel depicting plasmid DNA (“control”) incubated with isolated seminal plasma protein (“Sem Pl”; 1 mg/ml) following MSP-I/HIND-III restriction digest (“cut DNA”). The digest was stopped by heating at 65° C. prior to protein treatments. Bovine serum albumin (“BSA”) served as a negative control following restriction digest. DNase-I served as the positive control. rFAA (250 μg/ml) caused DNA degradation above and beyond that observed with seminal plasma treatments. A molecular weight marker (1 kb DNA; New England Biolabs) is shown in the far left lane.

FIG. 3 is a gel depicting the dose-response degradation of DNA exposed to rFAA (50 to 250 μg/ml) as compared to SP treatment alone. BSA and empty vector served as the negative controls. DNase-I served as the positive control. rFAA demonstrated DNase activity in a dose response manner. The activity observed using 50 μg/ml rFAA was higher than that achieved using whole seminal plasma protein extracts at 1 mg/ml.

FIG. 4 is a histogram showing that rFAA reduces binding of neutrophils to sperm. The percentage change in sperm-neutrophil binding is compared to control (no treatment). Sperm were incubated with the indicated treatments (see the Examples for details) and sperm-neutrophil binding was subsequently evaluated. DNase I, SP-Proteins (SP-P), and rFAA treatments significantly decreased binding between bovine sperm and neutrophils in vitro (P<0.001; ANOVA). There was no statistical difference between empty vector treatment and control. Purified rFAA (80 μg/ml) was as effective at inhibiting sperm-neutrophil binding as whole seminal plasma extracts used at 5-fold higher concentrations (400 μg/ml).

### DETAILED DESCRIPTION OF THE INVENTION

As used herein, the term “DNase” refers to any enzyme having DNA endonuclease activity, including, without limitation, enzymes falling within Enzyme Classification Nos. EC 3.1.21; EC 3.1.22; EC 3.1.25; EC 3.1.30; and EC 3.1.31. Explicitly included (without limitation) within the term “DNase” as used herein are enzymes commonly designated as follows:

EC 3.1.21.1 deoxyribonuclease I;

EC 3.1.21.2 deoxyribonuclease IV (phage-T4-induced);

EC 3.1.21.3 type I site-specific deoxyribonuclease;

EC 3.1.21.4 type II site-specific deoxyribonuclease;

EC 3.1.21.5 type III site-specific deoxyribonuclease;

EC 3.1.21.6 CC-preferring endodeoxyribonuclease;

EC 3.1.21.7 deoxyribonuclease V;

EC 3.1.22.1 deoxyribonuclease II;

EC 3.1.22.2 Aspergillus deoxyribonuclease K1;

EC 3.1.22.4 crossover junction endodeoxyribonuclease;

EC 3.1.22.5 deoxyribonuclease X;

EC 3.1.25.1 deoxyribonuclease (pyrimidine dimer);

EC 3.1.30.1 Aspergillus nuclease S1;

EC 3.1.30.2 Serratia marcescens nuclease; and

EC 3.1.31.1 micrococcal nuclease.

The term “DNase I-like protein” refers to an enzyme having an enzyme classification of EC 3.1.21.x, where “x” is a positive integer. As used herein, “DNase I-like proteins” are a subset of “DNases.” DNases are available from a host of international commercial suppliers, including New England BioLabs, Promega (Madison, Wis.), and many others.

The terms “reporter molecule” and “reporter moiety” are synonymous and are used herein to denote a molecule or moiety that can be bonded to, adhered, or otherwise affixed to a DNA molecule (by any type of bonding), and which generates a signal that can be tracked. Chromophores, fluorophores, radioactive reporters, metallic reporters, etc. are all types of reporter molecules. A host of such reporter molecules are available commercially. Invitrogen, of Carlsbad, Calif., is a long-time commercial supplier of a host of such reporter molecules. An exemplary (non-limiting list) of such reporter molecules includes fluorescein and fluorecein derivates (e.g., FITC, carboxyfluorescein, 5-chloromethylfluorescein), rhodamine and rhodamine derivatives (e.g., carboxy rhodamine, TAMRA) BODIPY, dabcyl, dansyl, ethidium bromide, HEX, SYBR, YOYO-1, YOYO-3 and the like.

Many of the steps noted below for manipulating DNA and enzymes, for digesting with restriction endonucleases, for separating and isolating by gel electrophoresis, and the like, are well known and widely practiced by those skilled in the art. These conventional procedures and are not extensively elaborated upon herein. Unless otherwise noted, the DNA and enzymatic protocols utilized herein are described extensively in Sambrook, J.; Fritsch, E. F.; Maniatis, T. (1989), Molecular Cloning: A Laboratory Manual; Cold Spring Harbor Laboratory Press: New York, N.Y.

The invention centers around the discovery that DNase activity correlates positively with semen fertility. While not being limited to any underlying biological phenomena, it is believed that increased DNase activity in semen leads to increased fertility by breaking down or otherwise interfering with neutrophil extracellular traps (“NETs”) created by neutrophils within the reproductive tract. These NETs are believed to impede sperm, thus reducing the overall fertility of the semen containing the sperm. Thus, as noted earlier, the invention is a method of gauging the fertility of semen from a given male individual by measuring DNase activity within the semen and comparing it to an arbitrary semen standard or other sample. The DNase activity can be determined using any method know known or developed in the future for measuring DNase activity in semen or semen extracts.

Likewise, the invention comprises a composition for storing semen and for improving its fertility by adding exogenous DNase to the solution in which the semen is stored. The DNase functions to improve the fertility of the semen, both when the semen is used fresh and when the semen is used after being cryopreserved. Similarly, the invention comprises a method of storing semen wherein a DNase is added to the semen prior to cryopreservation. (The DNase could also be added to thawed semen.)

The invention is also drawn to a method of contraception in mammals, the method comprising inhibiting DNase activity in semen. Here, the DNase activity in the semen is inhibited by contacting the semen with a fertility-compromising amount of a compound that binds specifically to DNase enzymes and inhibits the ability of the DNase enzymes to hydrolyze a DNA substrate. Any non-toxic DNase inhibitor now known or developed in the future may be used in the method. Several suitable DNase inhibitors are known, including aurintricarboxylic acid (available commercially from Calbiochem, La Jolla, Calif.), inhibitor of GzmA-activated DNase (see Fan et al. (2003) Cell 112:659-672), and DNase inhibitors isolated from Nicotiniana tabacum (see Szopa & Wagner (1980) Eur. J. Biochem. 111:211-215.

Another embodiment of the invention is a method of treating endometritis in mammals. The method comprising administering a NET-degrading-effective amount of a compound selected from the group consisting of DNase, FAA, and combinations thereof, to the uterus of a mammal. Insofar as a many uterine maladies, most notably endometritis, are characterized by the extensive formation of NETs, using DNase to degrade these NETs serves to ameliorate and/or inhibit the progress of endometritis and other uterine diseases that involve the formation of NETs within the uterus of a mammal.

### EXAMPLES

The following Examples are included to provide a more complete description of the invention disclosed and claimed herein. The Examples do not limit the scope of the invention in any fashion.

Recombinant FAA: Cloning, expression and purification of rFAA is described in detail in U.S. Pat. No. 6,891,029, issued May 10, 2005, which is incorporated herein by reference.

Seminal Plasma Protein Preparation: Proteins were precipitated from pooled bovine seminal plasma (SP) with ammonium sulfate (33% w/v). A saturated solution of ammonium sulfate was made by adding 530 g/L ammonium sulfate into double-distilled H₂O (ddH₂O). Ammonium sulfate was mixed with pooled SP drop-by-drop to a final concentration of 33%. The SP mixture was incubated at 4° C. with rocking for 30 min. Following incubation, the mixture was centrifuged at 3,000×g for 20 min at 4° C. to pellet precipitated protein. Precipitated protein was re-suspended in phosphate-buffered saline (PBS) equal to the initial volume of SP and containing 10 μM phenylmethanesulfonyl fluoride (PMSF) and 1 μM pepstatin A. SP proteins were dialyzed overnight at 4° C. using 10 kDa molecular weight cut-off “SnakeSkin”-brand dialysis tubing (Pierce, Rockford, Ill.) against a 50× volume of PBS containing PMSF and pepstatin A with at least two changes of buffer. Following dialysis, any precipitate was removed by centrifugation. Soluble protein was quantified using a bicinchoninic acid (BCA) protein assay (Pierce), following the manufacturer's instructions. Absorbance was determined using a Biophotometer (Eppendorf, Westbury, N.Y.). Protein concentration was calculated and purity of the SP protein extract was assessed by sodium dodecylsulfate-poly(acrylamide) gel electrophoresis (SDS-PAGE). Protein samples were aliquoted and stored at −20° C.

Preparation of Polymorphonuclear Neutrophils (“PMNs”): Blood was collected from a healthy cow in heparinized tubes, and PMNs were isolated with “PREMIUM”-brand Ficoll-Paque (GE Healthcare Bio-Sciences Corp., Piscataway, N.J.) following the manufacturer's instructions. Once collected, blood was diluted 1:1 with PBS and carefully layered over the Ficoll-Paque layer at a ratio of 4 ml diluted whole blood to 3 ml Ficoll-Paque. The mixture was centrifuged at 400×g for 30 min. at room temperature and upper layers were removed, leaving the bottom layer containing the red blood cells (RBC) undisturbed. The RBC pellet was re-suspended in 5 ml sterile ddH₂O for 45 sec to lyse RBC followed by the addition of PBS up to 50 ml. The lysed RBC mixture was centrifuged and the resulting pellet was re-suspended in 1 ml of PBS. The 1 ml of cells was layered over new Ficoll-Paque layer and centrifuged for 5 min at 400×g at room temperature to remove any cellular debris. The cell pellet was re-suspended in PBS, cell concentration was determined using a hemacytometer, and the pellet was then diluted to a final concentration of 14×10⁶. Neutrophils were stored on ice up for up to 3 h. Smears were made from isolated neutrophils and stained using Wright-Giemsa stains (HEMA 3®-brand Stain Set, Fisher Scientifics, Middletown, Va.) following the manufacturer's instruction to ensure neutrophils were isolated.

Sperm Preparation: Seminal fluid was removed from fresh semen by centrifugation and sperm pellets were washed (3×) and re-suspended in Tyrode's albumin-lactate-pyruvate media (“TALP media”). TALP media can be obtained commercially from several sources, including Millipore Specialty Media (Billerica, Mass.). Sperm concentration was determined and sperm samples were adjusted to a concentration of 50×10⁶ sperm cells/ml using TALP medium. Cryopreserved semen samples were thawed, washed (3× in TALP medium) and re-suspended in TALP at 50×10⁶ sperm cells/ml.

Sperm-Neutrophil Binding Assay and Evaluation: The effects of SP proteins and recombinant fertility-associated antigen (rFAA) on sperm binding to blood-derived neutrophils were determined after spermatozoa were first incubated with respective treatments (80 or 160 μg/ml rFAA; 200 or 400 μg/ml SP extracts) for 30 min at 38° C., followed by co-incubation with neutrophils under the same conditions. Sperm were incubated for 30 min with neutrophils and the extent of sperm-neutrophil binding was determined. Wet mounts of sperm binding to neutrophils were evaluated by light microscopy and expressed as the proportion of neutrophils that bound to at least one spermatozoon. A drop of the sperm-neutrophil mixture was placed on a glass slide, covered with a cover slip, and the number of sperm bound to neutrophils was determined using a Leica DMLS microscope (Bannockburn, Ill.) at 400× magnification. A minimum of 200 neutrophils were counted per slide.

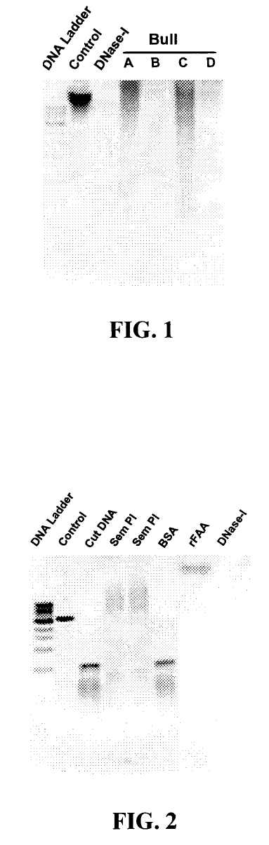

Detection of Endonuclease Activity in Seminal Plasma and rFAA: Endonuclease activities of crude SP protein extracts and rFAA were analyzed by standard agarose electrophoresis. Bovine pancreatic DNase I (0.25 μg/ml) or empty vector (a non-transformed cell line) were used as positive controls. The treatments comprised co-incubating the various combinations of additives in a reaction buffer (10 mM Tris-Cl, 2.5 mM MgCl₂, 0.5 mM CaCl₂ (pH 7.6)) containing 1 μg of DNA (pBR322; New England Biolabs, Ipswich, Mass. or calf thymus DNA; Sigma, St. Louis, Mo.) at 38° C. for 30 min. Dose response activity of rFAA was determined by adding 0, 50, 100, 150, 200 or 250 μg/ml to DNA substrate. To determine whether seminal DNase activity could be enhanced by rFAA, DNA was treated simultaneously with seminal plasma extracts (1 or 4 mg/ml) and rFAA (0, 50, or 100 μg/ml). Inactivation of SP and DNase I was performed by heating to 70° C. for 10 min before adding plasmid DNA. To determine whether DNase activity varied among bulls, samples of whole SP protein extracts from four (4) different bulls were compared to each other and to controls using 1 μg/ml of plasmid DNA as substrate in each treatment. The results are shown in FIG. 1.

As shown in FIG. 1, DNase activity in seminal plasma is measured by incubating an unknown sample to which is added a known quantity of DNA. The sample is then incubated for a fixed amount of time in a suitable buffer. The extent of DNA hydrolysis is then determined by conventional agarose gel electrophoresis followed by visualization of the DNA banding pattern. Samples with higher DNase activity are readily discernible based on the extent of DNA hydrolysis in a lane. That is, the band of DNA is extensively smeared, thus evidencing the increased activity of DNase, which functions to cleave each DNA molecule into two or more fragments. Males whose seminal plasma exhibits a higher level of DNase activity have higher fertility semen as compared to males whose plasma exhibits a lower level of DNase activity.

FIG. 1 depicts a typical assay showing the variation of DNase activity among seminal plasma protein extracts (SP-protein extracts) isolated from four individual bulls (designated as bulls A, B, C, D in FIG. 1. One μg of plasmid DNA (pBR322) was incubated with DNase-I (0.25 μg/ml; positive control) or seminal plasma extracts (1 mg/ml) from each bull. A 1 kb molecular weight marker is shown in the far left-hand lane and the “control” lane contains uncut plasmid DNA. The results depicted in FIG. 1 reveal that bulls A and C have higher-fertility sperm than bulls B and D.

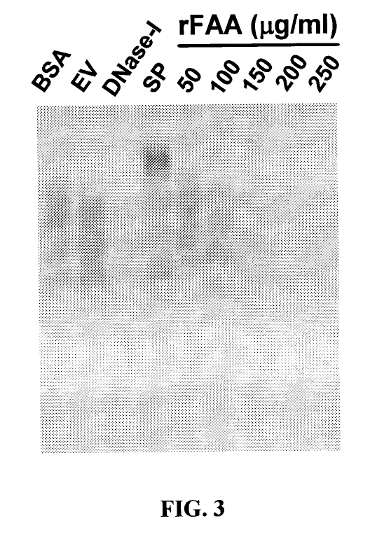

Importantly, FAA possesses two DNase-1-like signature motifs and a recombinant bovine FAA (rFAA) displays the ability to hydrolyze DNA in a dose-response manner in vitro (see FIGS. 2 and 3). In FIG. 2, plasmid DNA (control) was digested with the enzyme MSP-I/HIND-III to yield fragments (designated “cut DNA” in FIG. 2) followed by incubation with seminal plasma protein extracts (Sem Pl; 1 mg/ml) or rFAA (250 μg/ml). Following restriction digest, the reaction was stopped by heating at 65° C. (second Sem Pl lane). Bovine serum albumin (BSA) served as a negative control and DNase I was used as a positive control. A 1 kb DNA ladder is shown in the first lane. FIG. 2 shows that rFAA caused a very prominent upward shift in DNA migration. This indicates that rFAA partially degraded the supercoiled plasmid DNA. These findings are significant in that it was unknown that rFAA had DNase activity. Thus, FIG. 2 shows that exogenous FAA added to semen can increase the DNase activity of the semen.

FIG. 3 is a gel depicting dose-response degradation of DNA upon exposure to increasing concentrations of rFAA (50 to 250 μg/ml) compared to seminal plasma (SP) treatment alone. BSA and empty vector (EV) served as negative controls. DNase-I served as positive control. The significance of FIG. 3 is that it demonstrates not only that FAA has DNase activity, but also that this activity exerts itself in a dose-dependent fashion.

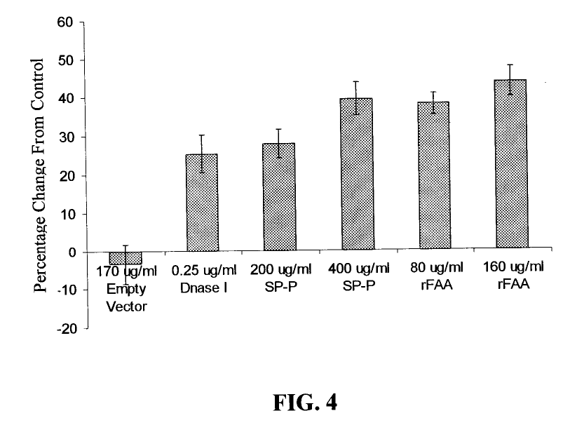

Purified rFAA also inhibits bovine sperm-neutrophil binding, as evidenced by the histogram shown in FIG. 4. FIG. 4 depicts the percentage change in sperm-neutrophil binding as compared to control (no treatment). To generate the data shown in FIG. 4, sperm were incubated with the indicated treatments (as described earlier) and sperm-neutrophil binding was subsequently evaluated. DNase I, SP-Proteins (SP-P), and rFAA treatments significantly decreased binding between bovine sperm and neutrophils in vitro (P<0.001; ANOVA). There was no statistical difference between empty vector treatment and control. Purified rFAA (80 μg/ml) was as effective at inhibiting sperm-neutrophil binding as whole seminal plasma extracts used at 5-fold higher concentrations (400 μg/ml)

Variation in seminal DNase activity exists among bulls (see FIG. 1). While not being tied to a particular biological mechanism, it is reasonable to conclude that differences in fertility are related, in part, to sperm-neutrophil interactions, which in turn are related to seminal DNase activity. The entire collection of data presented in these Examples suggests that differential seminal DNase activity can be accounted for by variation in FAA content.

Micro-Well Plate Assay: An alternative approach for assaying for DNase activity is to perform a calorimetric, fluorescent, radiographic, or other type of reporter molecule assay within a conventional vessel, such as a micro-well plate (e.g., 96-well plate, 384-well plate, etc.). Into each well of the plate is adhered or placed a known, fixed amount of DNA which has been labeled using any number of well-known biochemical reporter moieties (biotin, a chromophore, a fluorophore (e.g., fluorescein), colloidal gold, a radioactive label such as ³H or ¹⁴C, etc.). A large number of suitable reporter moieties for use in the present invention are commercially available from many suppliers, for example, from Molecular Probes, a division of Invitrogen (Carlsbad, Calif.). A base-signal is then measured. To each well, a fixed volume of seminal plasma from various mammalian males is added and the plate is incubated, the reactions are stopped, and the plates are gently washed. If hydrolysis occurs, the hydrolyzed DNA fragments are removed from the wells by the washing step. The signal through and “label” will decrease. The assay is quantifiable and scalable for high-throughput fertility analysis.

Cassette or Dot-Blot Assays: Colloidal gold-labeled DNA is immobilized on a device with a lateral-flow membrane. Semen samples are applied to the assay and as hydrolysis of DNA occurs, the gold signal would be predicted to diminish. Any suitable biochemical label of DNA would be applicable to this assay. Similarly, colloidal gold-labeled DNA could be spotted onto a dot blot. A semen sample would be added to the well, and dissolution of the gold would correspond to DNase activity of the semen sample added to the well. The assay is quantifiable and scalable for high-throughput fertility analysis.

It is understood that the invention is not confined to the particular construction and arrangement of parts herein illustrated and described, but embraces such modified forms thereof as come within the scope of the claims following the Bibliography.

### BIBLIOGRAPHY

Alghamdi A., Troedsson M. H., Laschkewitsch T. and J. L. Xue (2001) “Uterine secretion from mares with post-breeding endometritis alters sperm motion characteristics in vitro,” Theriogenology 55:1019-1028.
Alghamdi A. S., Foster D. N. and M. H. Troedsson (2004) “Equine seminal plasma reduces sperm binding to polymorphonuclear neutrophils (PMNs) and improves the fertility of fresh semen inseminated into inflamed uteri,” Reproduction 127:593-600.
Alghamdi, A. S. and D. N. Foster (2005) “Seminal DNase frees spermatozoa entangled in neutrophil extracellular traps,” Biol. Reprod. 73:1174-1181.
Brinkmann V., Reichard U., Goosmann C., Fauler B., Uhlemann Y., Weinrauch Y., Zychlinsky A, and D. S. Weiss (2004) “Neutrophil extracellular traps kill bacteria,” Science 303:1532-1535.
Kaeoket K., Persson E. and A. M. Dalin (2003) “Influence of pre-ovulatory insemination and early pregnancy on the infiltration by cells of the immune system in the sow endometrium,” Anim. Reprod. Sci. 75:55-71.
McCauley T. C., Zhang H., Bellin M. E. and R. L. Ax (1999) “Purification and characterization of fertility-associated antigen (FAA) in bovine seminal fluid,” Mol. Reprod. Dev. 54:145-153.
Robertson, S. A. (2007) “Seminal fluid signaling in the female reproductive tract: lessons from rodents and pigs,” J. Anim Sci. 85 (13 suppl.) E36-44.
Rozeboom K. J., Toedsson M. H. and B. G. Crabo (1998) “Characterization of uterine leukocyte infiltration in gilts after artificial insemination,” J. Reprod. Fertil. 114:195-199.
Rozeboom K. J., Troedsson M. H., Hodson H. H., Shurson G. C. and B. G. Crabo (2000) “The importance of seminal plasma on the fertility of subsequent artificial inseminations in swine,” J. Anim. Sci. 78:443-448.
Rozeboom K J, Troedsson M H, Rocha G R and B. G. Crabo (2001) “The chemotactic properties of porcine seminal components toward neutrophils in vitro,” J. Anim. Sci. 79: 996-1002.
Sato F., Soh T., Hattori M. A. and N. Fujihara (2003) “Evaluation of deoxyribonuclease activity in seminal plasma of ejaculated chicken semen,” Asian J. Androl. 5:213-216.
Shastina V. V., Menzorova N. I., Sibirtsev Y. T. and V. A. Rasskazov (2003) “Purification and characteristics of Ca²⁺,Mg²⁺- and Ca²⁺,Mn²⁺-dependent and acid DNases from spermatozoa of the sea urchin Strongylocentrotus intermedius,” Biochemistry (Mosc) 68:582-592.
Tremellen K. P., Seamark R. F. and S. A. Robertson (1998) “Seminal transforming growth factor beta1 stimulates granulocyte-macrophage colony-stimulating factor production and inflammatory cell recruitment in the murine uterus,” Biol. Reprod. 58:1217-1225.
Troedsson M. H. T., K. Loset, A. M. Alghamdi, B. Dahms, and B. G. Crabo (2001) “Interaction between equine semen and the endometrium: the inflammatory response to semen,” Anim. Reprod. Sci. 68:273-278.
Troedsson, M. H. T., I. K. Liu, and B. G. Crabo (1998) “Sperm transport and survival in the mare: a review,” Theriogenology 50:807-818.
Urban C. F., Reichard, U., Brinkmann, V. and A. Zychlinsky (2006) “Neutrophil extracellular traps capture and kill Candida albicans yeast and hyphal forms,” Cell Microbiol. 8:66-8-676.
Yasuda T., Sawazaki K., Nadano D., Takeshita H., Nakanaga M., K. Kishi (1993) “Human seminal deoxyribonuclease I (DNase I): purification, enzymological and immunological characterization and origin,” Clin. Chim. Acta 218:5-16.

What is claimed is:

1. A method for measuring mammalian semen fertility, the method comprising measuring DNase activity in a semen sample, wherein greater DNase activity in the semen sample as compared to a control sample indicates increased fertility of the semen sample.

2. The method of claim 1, comprising measuring DNase activity in the semen sample by concentrating seminal plasma proteins from the sample to yield seminal plasma protein extract, spiking the seminal plasma protein extract with a known amount of DNA to yield DNA-spiked seminal plasma protein extract, and measuring DNA hydrolysis in the DNA-spiked seminal plasma protein extract, wherein an increased level of DNA hydrolysis indicates an increased level of DNase activity in the semen sample.

3. The method of claim 2, wherein the level of DNA hydrolysis is measured via gel electrophoresis.

4. The method of claim 1, wherein the activity of DNase I is measured.

5. The method of claim 1, wherein the activity of a DNase I-like protein is measured.

6. A method for enhancing the fertility of mammalian semen, the method comprising adding exogenous DNase, exogenous fertility-associated antigen (FAA), or both DNase and FAA to semen.

7. The method of claim 6, wherein the DNase, the FAA, or the DNase and FAA are added to the semen prior to cryopreserving the semen.

8. The method of claim 6, further comprising cryopreserving the semen after the DNase, the FAA, or the DNase and FAA are added to the semen.

9. The method of claim 6, wherein from about 0.001 μg/mL to about 1.0 μg/mL of exogenous DNase is added to the semen or from about 10 to about 100 μg/mL of FAA is added to the semen.

10. The method of claim 6, wherein the exogenous DNase added to the semen is a DNase I enzyme, EC 3.1.21.1.

11. The method of claim 6, wherein the exogenous DNase added to the semen is a DNase I-like protein, EC 3.1.21.x.

12. A method of storing semen for future use in artificial insemination, the method comprising:
(a) adding a fertility-enhancing-effective amount of exogenous DNase, or exogenous fertility-associated antigen (FAA, or both DNase and FAA to the semen; and then
(b) cryopreserving the semen from step (a).

13. The method of claim 12, wherein in step (a) from about 0.001 μg/mL to about 1.0 μg/mL of the exogenous DNase is added to the semen or from about 10 to about 100 μg/mL of FAA is added to the semen.

14. The method of claim 12, wherein the exogenous DNase added to the semen is a DNase I enzyme, EC 3.1.21.1.

15. The method of claim 12, wherein the exogenous DNase added to the semen is a DNase I-like protein, EC 3.1.21.x.

16. The method of claim 12, wherein the semen in step (a) is disposed in a cryopreservation media.

17. The method of claim 16, wherein the cryopreservation media is selected from the group consisting of citrate-based milk extenders and Tris-based egg yolk extenders.

18. A composition of matter for increasing fertility of semen, the composition comprising a diluent and an amount of exogenous DNase, an amount of exogenous fertility-associated antigen (FAA), or an amount of both DNase and FAA disposed in the diluent, wherein the amount of the exogenous DNase, exogenous FAA, or DNase and FAA is effective to increase fertility of the semen.

19. The composition of matter of claim 18, wherein the diluent is Tyrode's albumin-lactate-pyruvate media.

20. The composition of claim 18, wherein the exogenous DNase is a DNase I enzyme, EC 3.1.21.1.

21. The composition of claim 18, wherein the exogenous DNase is a DNase I-like protein, EC 3.1.21.x.

22. The composition of claim 18, wherein the amount of exogenous DNase yields a solution having a concentration of from 0.001 μg/mL to about 1.0 μg/mL exogenous DNase or from about 10 to about 100 μg/mL of FAA is added to the semen.

23. The composition of claim 18, further comprising sperm disposed within the semen.

24. A method of contraception in mammals, the method comprising inhibiting DNase activity in semen.

25. The method of claim 24, wherein the DNase activity is inhibited by contacting the semen with a compound that binds specifically to DNase enzymes and inhibits the ability of the DNase enzymes to hydrolyze a DNA substrate.

26. A method of treating endometritis in mammals, the method comprising administering a NET-degrading-effective amount of a compound selected from the group consisting of DNase, FAA, and combinations thereof, to the uterus of a mammal.
